(19) United States
(12) Patent Application Publication
(10) Pub. No.: US 2012/0197387 A1
(43) Pub. Date: Aug. 2, 2012

(54) STENT WITH INTERNAL FORCE AND FABRICATION METHOD THEREOF

(75) Inventors: Kwang Koo JEE, Seoul (KR); Jun Hyun HAN, Seoul (KR)

(73) Assignee: KOREA INSTITUTE OF SCIENCE AND TECHNOLOGY, Seoul (KR)

(21) Appl. No.: 13/352,473

(22) Filed: Jan. 18, 2012

(30) Foreign Application Priority Data
Feb. 1, 2011 (KR) 10-2011-0010335

(51) Int. Cl. A61F 2/82; C21D 1/70

(52) U.S. Cl. 623/1.46; 148/537

(57) ABSTRACT

A stent includes: a main body unit including a plurality of through holes and configured to have a net-like shape; and a coating unit filling the through holes of the main body unit and configured as a layer on the main body unit, wherein the main body unit has force of restitution to be restored to its original shape, and the coating unit includes a support unit for maintaining the shape of the main body unit by hampering the restoration of the main body unit and a blocking unit blocking the through holes of the main body unit to discriminate the interior and the exterior of the main body unit. The stent can be quickly expanded in a blood vascular system, or the like, after being inserted into the body in a surgical procedure, thus improving expansion (or extension) effect.

### FIELD OF THE INVENTION

The present invention relates to a stent and, more particularly, to a stent having improved force of restitution.

### DESCRIPTION OF THE RELATED ART

In general, a stent refers to a medical tool inserted into a body part, which has been narrowed or contracted due to a malicious or a benign disease such as blood vessels, gastrointestine, a biliary tract, or the like, obstructing a smooth flow of blood or body fluids therein, in order to normalize the flow of blood, or the like, under radioscopy without surgery.

When the stent is inserted into a narrowed or contracted body part, the stent contracts in shape at an initial stage so as to be inserted, and then, after the stent is inserted and an external force eliminated. Then, the contracted stent is restored into its original shape to expand a blocked or narrowed part of blood vessels, gastrointestine, a biliary tract, or the like.

The stent has a tube-like shape in a metal wire net, and is divided into a balloon expandable stent made of cobalt-chromium (Co—Cr) or stainless alloy and a self-expandable stent made of a nickel-titanium (Ni—Ti)-based shape memory alloy.

The self-expandable stent made of a nickel-titanium-based shape memory alloy has advantages in that it is simply applicable, has excellent expandability, and expands with a regular extensionality, and thus, the self-expandable stent is commonly used for a blood vascular system procedure and non-vascular system procedure.

FIG. 1 is a graph showing a deformation behavior of a general metal, and FIG. 2 Is a graph showing a deformation behavior of a nickel-titanium-based shape memory alloy.

With reference to FIG. 1, a general metal such as stainless steel, or the like, may be permanently deformed. Thus, when a stent is made of a general metal and inserted into blood vessels or the like, the stent may not completely spread, and although it spreads, the force is significantly reduced. Thus, the stent made of a general metal may be used only as a balloon-expandable stent.

With reference to FIG. 2, the nickel-titanium-based shape memory alloy is only elastically deformed. Thus, when a stent is made of the nickel-titanium-based shape memory alloy and inserted into vessels, or the like, the stent can completely spread. Also, as shown in the graph of a deformation rate versus stress in FIG. 2, due to the section in which there is little change in force according to a deformation rate, a narrowed or blocked portion of the blood vascular system in the body, or the like, can be expanded within a short time without an additional expansive force (stress).

However, even in this case, the stent has a section in which there is much change in force according to the deformation rate, so at this section, the stent expands and the expansive force is rapidly reduced.

Thus, from this time, the speed of expanding and treating intestine is slowed down. Thus, when the stent is returned to its original shape, the expansive force does not work any longer.

In this case, a thick wire may be used or the alloy composition or thermal treatment conditions may be improved to increase the tilt of the curve of the expansive force, but this cannot solve the foregoing problems, and this is the same to the stent made of a general metal.

### SUMMARY OF THE INVENTION

Therefore, in order to address the above matters, the various features described herein have been conceived.

An aspect of the present invention provides a stent capable of being quickly expanded in a blood vascular system, or the like, after being inserted into the body in a surgical procedure to thus improve expansion (or extension) effect.

According to an aspect of the present invention, there is provided a stent including: a main body unit including a plurality of through holes and configured to have a net-like shape; and a coating unit filling the through holes of the main body unit and configured as a layer on the main body unit, wherein the main body unit has force of restitution to be restored to its original shape, and the coating unit includes a support unit for maintaining the shape of the main body unit by hampering the restoration of the main body unit and a blocking unit blocking the through holes of the main body unit to discriminate the interior and the exterior of the main body unit.

The main body unit of the stent may be made of a shape memory alloy.

The coating unit of the stent may be configured as a tube covering the main body unit.

The coating unit of the stent may be coated on the main body unit.

According to another aspect of the present invention, there is provided a method for fabricating a stent, including: forming a main body unit such that it has a certain shape by using a nickel-titanium material; thermally treating the main body unit to provide characteristics of a shape memory alloy to the main body; contracting the main body unit to have a diameter of a stent desired to be fabricated and fixing the contracted diameter; and forming a coating unit on the main body unit.

In forming the shape of the main body unit, the main body unit may be formed by using any one of a method of weaving the nickel-titanium material in the form of a wire and a method of cutting the nickel-titanium material by a laser.

The forming of the coating unit may include: forming a tube corresponding to the diameter of a stent desired to be fabricated; and inserting the main body unit into the tube.

The forming of the coating unit may be performed by coating silicon rubber on the main body unit.

In the stent and the method for fabricating a stent according to embodiments of the present invention, after the main body unit of the stent is formed and contracted by applying external force thereto, the contracted shape of the main body unit is maintained, whereby the stent can have initial force, exhibits excellent expandability in the surgical procedure, and high fatigue life.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a graph showing a stress-deformation curve of general metal;

FIG. 2 is a graph showing a stress-deformation curve of a shape memory alloy;

FIG. 3 is a perspective view of a stent according to an embodiment of the present invention;

FIG. 4 illustrates a deformation state when the stent is expanded and contracted according to an embodiment of the present invention, wherein FIG. 4a shows the expanded stent and FIG. 4b shows the contracted stent;

FIG. 5 is a graph showing a relation between expensive force and the diameter of a shape memory alloy stent according to an embodiment of the present invention;

FIG. 6 is a flow chart illustrating a process of fabricating the shape memory alloy stent according to an embodiment of the present invention;

FIG. 7 is a graph explaining the principle of fabricating the stent according to an embodiment of the present invention;

FIG. 8 is a graph showing a change in the diameter of the stent having initial force and that of the stent without initial force over a change in force;

FIG. 9 is a graph showing the influence of a repeated deformation rate affecting a fatigue life of the stent; and

FIG. 10 is a graph showing an expansive-displacement curve of the stent of Comparative Example and that of Embodiment.

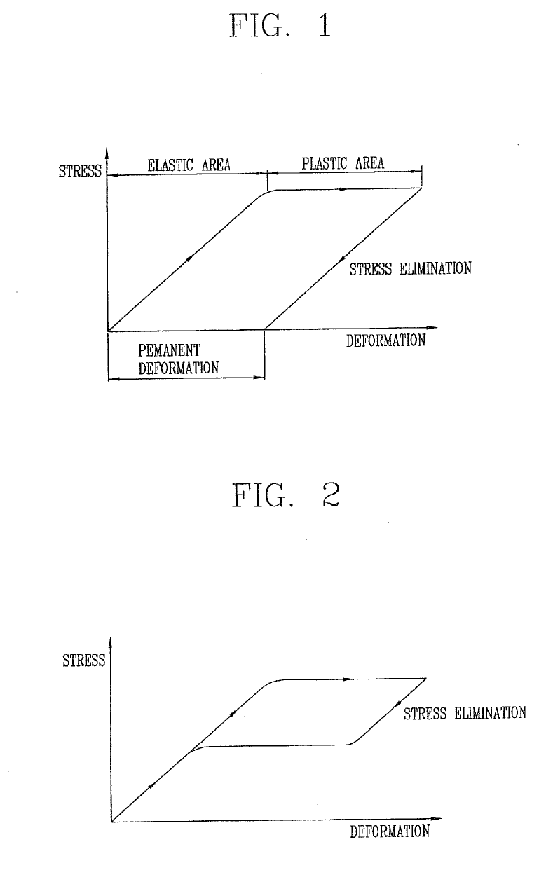

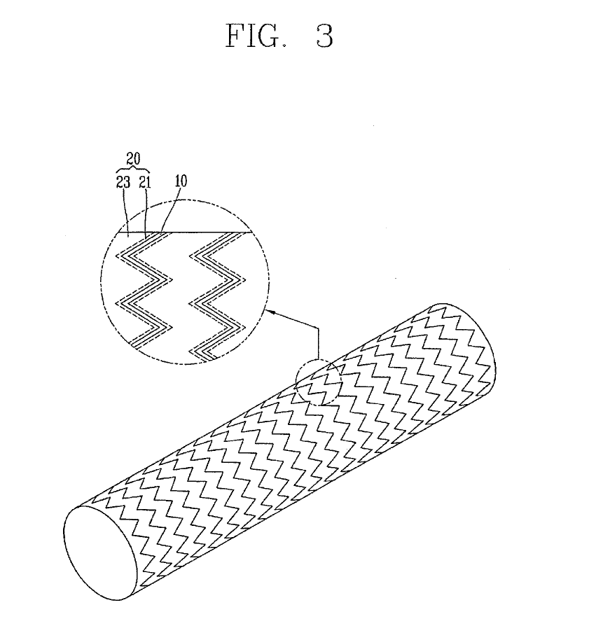

### DETAILED DESCRIPTION OF THE INVENTION

The above and other objects, features and advantages of the present invention will be more clearly understood from the following detailed description taken in conjunction with the accompanying drawings so that they can be easily practiced by those skilled in the art to which the present invention pertains. The present invention may be modified variably and may have various embodiments, particular examples of which will be illustrated in drawings and described in detail. However, it should be understood that the following exemplifying description of the invention is not intended to restrict the invention to specific forms of the present invention but rather the present invention is meant to cover all modifications, similarities and alternatives which are included in the spirit and scope of the present invention. The terms used in the present application are merely used to describe particular embodiments, and are not intended to limit the present invention.

A stent according to an embodiment of the present invention will now be described in detail with reference to the accompanying drawings.

FIG. 3 is a perspective view of a stent according to an embodiment of the present invention.

With reference to FIG. 3, a stent according to an embodiment of the present invention includes a main body unit 10 and a coating unit 20, and the coating unit 20 includes a support unit 21 and a blocking unit 23.

The main body unit 10 includes a plurality of through holes and has a net-like shape. The main body unit 10 is contracted in its initial shape and the contracted state of the main body unit 10 is fixed by the coating unit 20, so the main body unit 10 has force of restitution to return to the initial shape.

Preferably, the main body unit 10 is made of a shape memory alloy. When the main body unit 10 is made of a shape memory alloy, it can be self-expansive and have high restoration capability without causing plastic deformation.

Thus, since the main body unit 10 is configured as described above, the main body unit has so-called initial force required when it starts to contract so as to be mounted within a blood vessel. Accordingly, after the main body unit 10 is completely contracted, when it is expanded, expansive force acts until when the main body unit 10 completely spreads.

The coating unit 20 fills the through holes of the main body unit 10 and is formed as a layer on the main body 10. The coating unit 20 includes a support unit 21 maintaining the shape of the main body unit 10 by hampering the main body unit 10 from being restored and a blocking unit 23 blocking the through holes of the main body unit 10 to block the interior and the exterior of the main body unit.

The support unit 21 is configured as a film directly in contact with the main body unit 10, and formed to hamper the force of restitution of the main body unit 10.

The blocking unit 23 is connected with the support unit 21 and formed as a film to fill the through holes of the main body unit 10. The blocking unit 23 hampers the force of restitution of the main body unit 10 and prevents a cancerous tissue, or the like, from entering into the stent in cooperation with the support unit 21.

The coating unit 20 may be made of a material, such as silicon rubber, polytetrafluoreethylene (PTFE), a polycarbonate urethane, or the like, which has elasticity and is not harmful to a human body.

The main body unit 10 and the coating unit 20 of the stent according to an embodiment of the present invention will be supplementarily described as follows.

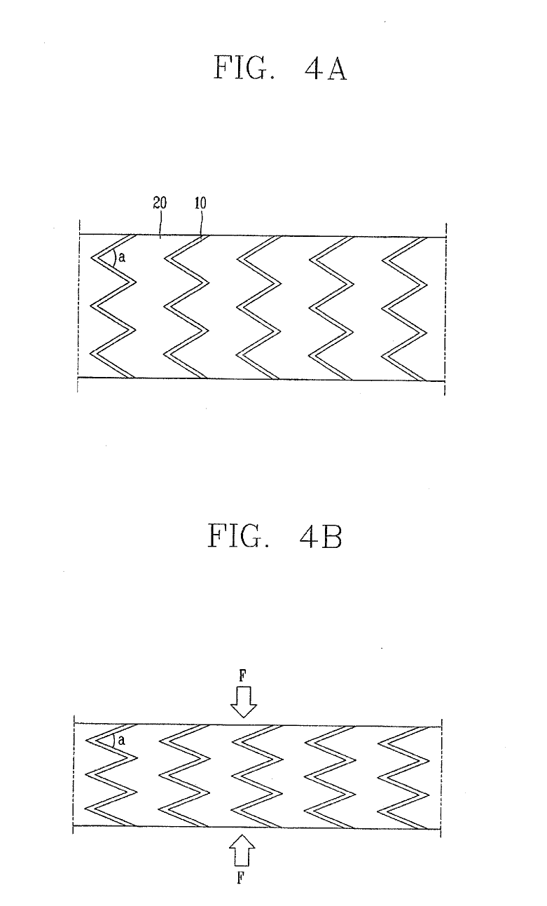

FIG. 4 illustrates a deformation state when the stent is expanded and contracted according to an embodiment of the present invention, wherein FIG. 4a shows the expanded stent and FIG. 4b shows the contracted stent.

With reference to the drawings, the stent according to an embodiment of the present invention uses elasticity according to bending. When force (F) is applied to the stent and the stent contracts, the angle (a) of the bending is reduced, and when the stent expands, the angle (a) is increased.

Here, when the stent is configured to include only the main body unit 10, although external force is applied to the main body unit 10 to contract the stent, when the external force is eliminated, the stent expands again so as to be restored.

Thus, it is difficult for the main body 10 having the foregoing force of restitution to have initial force by itself.

In the present embodiment, after the stent is contracted to have a smaller diameter by applying external force to the main body unit 10, the stent is supported such that it cannot be returned to the original state, thus allowing the stent to retain force to be expanded and thus providing initial force to the stent.

To this end, the elastic deformation of the main body 10 is prevented from being returned to the original state by using the coating unit 20, thus providing initial force.

Namely, in order to provide the initial force, an element for supporting the initial force is required and it can be achieved by the coating unit 20.

For example, when a thin tube made of silicon rubber is used as the coating unit 20, not much force is required to reduce the diameter of the tube, but a great amount of force is required to increase the diameter of the tube. Namely, the tube is able to serve as the support unit 21.

When the original diameter of the main body unit 10 (i.e., the diameter of the main body unit 10 not in a contracted state) is equal to an inner diameter of the rubber tube, the rubber tube scarcely affects the elasticity behavior of the main body unit 10. However, when the main body unit 10 which is compressed to a degree is covered with an appropriate rubber tube, the main body unit 10 cannot expand and only force for expansion will act.

At this time, the force for the main body unit 10 to expand and the force for the rubber to support the main body unit 10 are balanced.

In the above description, the tube made of silicon rubber is used as the coating unit 20, but a coating may be directly formed on the main body unit 10.

Hereinafter, the characteristics of the stent according to an embodiment of the present invention when the main body unit 10 is made of a shape memory alloy will be described in detail with reference to the accompanying drawings.

FIG. 5 is a graph showing a relation between expensive force and the diameter of a shape memory alloy stent according to an embodiment of the present invention.

With reference to FIG. 5, the shape memory alloy stent as described above does not have a significant change in the expansive force from a contracted state in which the stent has a diameter (B) to a state in which the stent is restored to have the original diameter (A), and the main body unit 10 expands up to a point (C) at which the force for the main body unit 10 to expand and the force for the coating unit 20 to support the main body 10 is equal.

Here, the portion indicated by the dotted line indicates a relationship between the expansive force and the diameter of the stent when the coating unit 20 is not provided. The main body unit 10 expands until when its expansive force is exhausted, and at this time, the main body unit 10 has the largest diameter.

A method for fabricating the shape memory alloy stent according to an embodiment of the present invention will now be described with reference to the accompanying drawings.

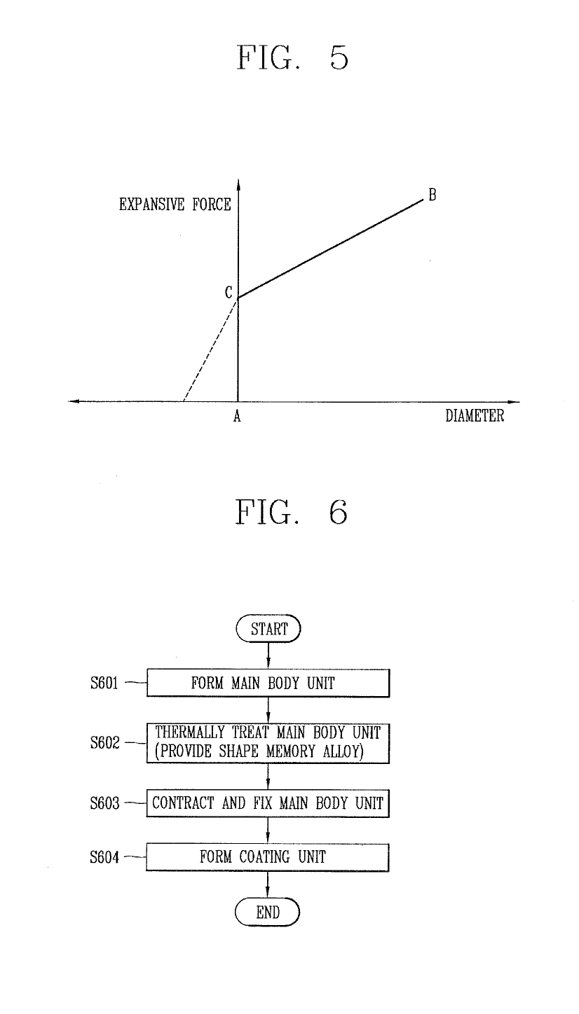

FIG. 6 is a flow chart illustrating a process of fabricating the shape memory alloy stent according to an embodiment of the present invention, and FIG. 7 is a graph explaining the principle of fabricating the stent according to an embodiment of the present invention.

First, the main body unit 10 is configured to have a form of a wire by using a nickel-titanium material or cut by a laser so as to be formed (S601).

Next, the main body unit 10 is fixed and thermally treated at 400° C. to 550° C. for a few minutes or a few hours (S602). Through the thermal treatment, the main body unit 10 can completely memorize a current shape thereof as a shape memory alloy, and excellent elasticity can be provided to the main body unit 10.

When the main body unit 10 is thermally treated to memorize the current shape thereof, the diameter after the thermal treatment is an initial diameter of the main body unit 10, and in this state, the main body unit 10 does not have force to expand or contract. The diameter of the main body unit 10 in this state is D2.

The expansive force measured while reducing the diameter of the main body unit 10 made of a nickel-titanium material fabricated as described above has a tendency as shown in the curve 1 in the graph of FIG. 7.

With reference to the curve 1 in the graph of FIG. 7, when the main body unit 10 contracts, force, starting from a state in which there is no force at the initial stage, is sharply increased according to a change in the distance between atoms (section 1a).

Thereafter, elasticity appears due to a generation of martensite, and it is noted that the force is gently increased (section 1b). An initial diameter of the main body unit 10 in this section is D1.

Thus, by contracting the main body unit 10 having the diameter D2 such that the main body unit 10 has the diameter D1, and then, the main body unit 10 is fixed to have the diameter D1 (S603).

Thereafter, the coating unit 20 is formed (S604). In forming the coating unit 20, the previously formed coating unit 20 may be put on the main body unit 10 or the coating unit 20 is coated on the main body unit 10.

The main body unit 10 tends to expand to have the original diameter D2, but when the coating unit 20 is formed thereon, the main body unit 10 is restrained from expanding, so the main body unit 10 has initial force Fi.

Resultantly, the curve 1 in the graph of FIG. 7 is equally matched to the curve 2 in the graph of FIG. 7, which indicates a force-diameter curve of the stent having the initial diameter D1, when it is moved to the left.

The characteristics and effect of the shape memory alloy stent according to an embodiment of the present invention will now be described in detail with reference to the accompanying drawings.

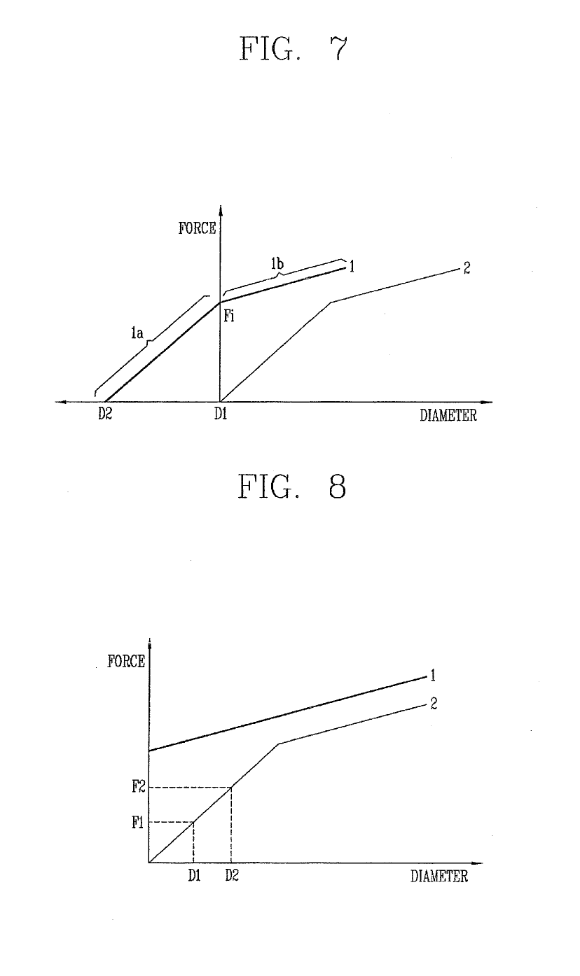

FIG. 8 is a graph showing a change in the diameter of the stent having initial force and that of the stent without initial force over a change in force.

The shape memory alloy stent according to an embodiment of the present invention has initial force, so it can quickly expands contracted intestine to allow for a fast medical treatment and increase a fatigue life.

In general, when the intestines such as blood vessel, large intestine, or the like, repeatedly receive force, the diameter of the stent may be significantly changed.

Namely, when the force is repeatedly changed by the sizes of F1 and F2, the diameter of the stent is repeatedly changed by D1 and D2. As a result, fatigue failure may take place.

In comparison, in the case of the stent having appropriate initial force, although external force is changed, if the external force is smaller than the initial force of the stent, the stent is not deformed, thus having a less possibility in which the fatigue failure occurs.

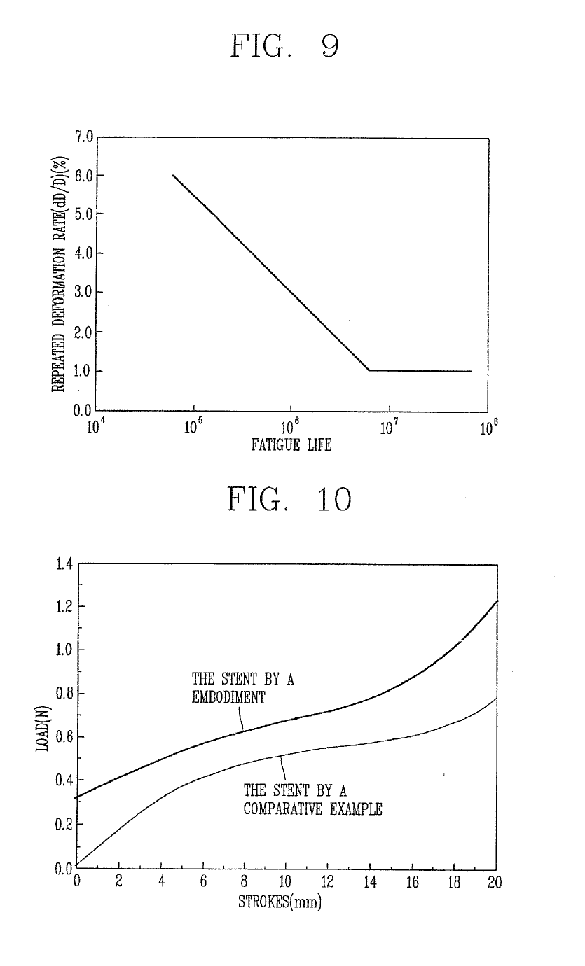

FIG. 9 is a graph showing the influence of a repeated deformation rate affecting a fatigue life of the stent.

As shown in FIG. 9, the fatigue life greatly depends on the deformation rate which is repeatedly occurs, and when the deformation rate is high, the fatigue life is short, while when the deformation rate is too low, the fatigue life is infinite.

Hereinafter, Embodiment of the present invention will be described in detail by comparing it with Comparative Example. The embodiments are merely proposed to understand the present invention and the scope of the present invention is not limited thereto and the embodiments include modifications, replacements, insertions, or the like, generally known in the art, and these are also included in the coverage of the present invention.

### Embodiment

A line material having a 50.8 nickel-titanium composition and having a diameter of 0.15 mm is twisted to have a diameter of 36 mm to form the main body unit 10, and then, the main body unit 10 was thermally treated at 500° C. for 10 minutes. In this state, although the main body unit 10 is compressed to be completely distorted, when the force is eliminated, the main body unit 10 is returned to the original state having the diameter 36 mm. The main body unit 10 was contracted to have a diameter of 24 mm and fixed. This is because the main body unit 10 tends to be returned to the original shape having the diameter of 36 mm. While rotating the main body unit 10, a silicon rubber solution was applied to the interior of the main body unit 10 in order to allow the silicon rubber solution to be uniformly coated on the main body unit 10 by centrifugal force. The silicon rubber was coated to have a thickness of 0.06 mm on the main body unit 10, dried, and then, a device for fixing the main body unit 10 was removed to obtain the shape memory alloy stent having a diameter of 24 mm.

### Comparative Example

A line material having a 50.8 nickel-titanium composition and having a diameter of 0.15 mm is twisted to have a diameter of 24 mm to form the main body unit 10, and then, the main body unit 10 was thermally treated at 500° C. for 10 minutes. Through the thermal treatment, the main body unit 10 can completely memorize a current shape thereof as a shape memory alloy, and excellent elasticity can be provided to the main body unit 10. In this state, although the main body unit 10 is compressed to be completely distorted, when the force is eliminated, the main body unit 10 is returned to the original state having the diameter 24 mm. While rotating the main body unit 10, a silicon rubber solution was applied to the interior of the main body unit 10 in order to allow the silicon rubber solution to be uniformly coated on the main body unit 10 by centrifugal force. The silicon rubber was coated to have a thickness of 0.06 mm and then dried to obtain a shape memory alloy stent having a diameter of 24 mm.

The respective stents fabricated through the different methods were compressed, and FIG. 10 shows the results. The stent according to Comparative Example has expensive force that increases, starting from a zero point, according to a reduction in the diameter. In comparison, it is noted that the stent according to Embodiment of the present invention requires initial force starting from an initial stage of compression.

When the stents are completely contracted and then expand in a body, the stent according to Comparative Example has expansive force which is drastically reduced as the stent expands, while the stent according to Embodiment of the present invention exhibits high expansive force until when the stent completely expands, having the effect that it can quickly open the intestine for a treatment.

When the fatigue life of the stent is measured, stress is repeatedly applied to the stent to have a deformation rate of ΔD/D=3.5% (D: diameter of stent and ΔD is a changed value of the diameter).

The diameter of a blood vessel of a healthy, young person is about ΔD/D=6.0% when the blood vessel is contracted or expanded, and when the stent is installed, it has a change of about 2-3.5%, so the deformation rate of about 3.5% is applied in many cases.

In order to repeatedly deform 0.8 mm corresponding to 3.5% of the diameter of 24 mm of the stent, force of about 0.07 N is required. The stent according to the Embodiment of the present invention is not deformed with this force.

Namely, when the existing stent is installed within a body (e.g., within the blood vessel), the stent according to Comparative Example is repeatedly deformed about 3.5%, but the stent according to Embodiment of the present invention does not deformed because it requires initial force.

Without the deformation rate, no fatigue failure occurs, and accordingly, of course, the fatigue life is improved.

As the present invention may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

What is claimed is:

1. A stent comprising:
a main body unit including a plurality of through holes and configured to have a net-like shape; and
a coating unit filling the through holes of the main body unit and configured as a layer on the main body unit,
wherein the main body unit has force of restitution to be restored to its original shape, and the coating unit includes a support unit for maintaining the shape of the main body unit by hampering the restoration of the main body unit and a blocking unit blocking the through holes of the main body unit to discriminate the interior and the exterior of the main body unit.

2. The stent of claim 1, wherein the main body unit is made of a shape memory alloy.

3. The stent of claim 1, wherein the coating unit is configured as a tube covering the main body unit.

4. The stent of claim 1, wherein the coating unit is coated on the main body unit.

5. A method for fabricating a stent, the method comprising:
forming a main body unit such that it has a certain shape by using a nickel-titanium material;
thermally treating the main body unit to provide characteristics of a shape memory alloy to the main body;
contracting the main body unit to have a diameter of a stent desired to be fabricated and fixing the contracted diameter; and
forming a coating unit on the main body unit.

6. The method of claim 5, wherein, in forming the shape of the main body unit, the main body unit is formed by using any one of a method of weaving the nickel-titanium material in the form of a wire and a method of cutting the nickel-titanium material by a laser.

7. The method of claim 5, wherein the forming of the coating unit comprises forming a tube corresponding to the diameter of a stent desired to be fabricated, and inserting the main body unit into the tube.

8. The method of claim 5, wherein the forming of the coating unit is performed by coating silicon rubber on the main body unit.
